(19) United States
(12) Patent Application Publication
(10) Pub. No.: US 2024/0269123 A1
(43) Pub. Date: Aug. 15, 2024

(54) MUSCLE REGENERATION PROMOTER

(71) Applicants: SATO PHARMACEUTICAL CO., LTD., Tokyo (JP); Keio University, Tokyo (JP)

(72) Inventors: Kazumasa OKUBO, Shinagawa-ku, Tokyo (JP); Yoshiki ITOH, Shinagawa-ku, Tokyo (JP); Masaya NAKAMURA, Shinjuku-ku, Tokyo (JP); Osahiko TSUJI, Shinjuku-ku, Tokyo (JP); Keisuke HORIUCHI, Shinjuku-ku, Tokyo (JP)

(73) Assignees: SATO PHARMACEUTICAL CO., LTD., Tokyo (JP); Keio University, Tokyo (JP)

(21) Appl. No.: 18/565,384

(22) PCT Filed: May 27, 2022
(86) PCT No.: PCT/JP2022/021745

§ 371 Date: Nov. 29, 2023

(30) Foreign Application Priority Data
May 31, 2021 (JP) 2021-091586

(51) Int. Cl. A61K 31/4439; A61K 31/417; A61P 21/00

(52) U.S. Cl. CPC A61K 31/4439; A61K 31/417; A61P 21/00

(57) ABSTRACT

The present invention is to provide a means for promoting muscle regeneration from muscle damage.

According to the present invention, the muscle regeneration from muscle damage is promoted by using a compound having histamine H receptor agonist activity (except for histamine) or a salt thereof is used as an active ingredient.

### TECHNICAL FIELD

The present invention relates to a muscle regeneration promoter comprising a compound having histamine H₃ receptor agonist activity (except for histamine) or a pharmaceutically acceptable salt thereof as an active ingredient.

### BACKGROUND ART

Muscle damage is clinically classified into those caused by muscle strain (pulled muscle), high-energy injury, surgical operation, and the like. Muscle damage is known to cause various complications (such as dysfunction, muscular atrophy, and local pain) (Non Patent Literature 1).

In such muscle damages, the muscle strain caused by blunt external force (bruise) is sports injury caused most frequently (Non Patent Literature 2). Although there are no accurate statistics, it is considered that the number of patients in Japan is around tens of thousands per year.

As the first aid for muscle damage, RICE treatment (Rest, Icing, Compression, and Elevation) has been recommended so far. After the first aid, symptomatic treatment for pain, rehabilitation, and the like have been conducted.

Muscle tissue has a mechanism for regeneration from the damage. It is known that muscle satellite cells are essential for muscle regeneration. The muscle satellite cells that reside in the vicinity of the muscle-fiber basement membrane, exist in a quiescent state under normal conditions, but when muscle damage is caused, the cells are activated and differentiated into myoblasts, and form muscle fibers via cell fusion. When the muscle regeneration is completed, the remaining muscle satellite cells enter the quiescent state again (Non Patent Literatures 3 and 4).

As the factor that promotes muscle regeneration, a hepatocyte growth factor (HGF) (Non Patent Literature 5) and an insulin-like growth factor 1 (IGF-1) (Non Patent Literature 6) are known. Further, it has been reported that muscle hypertrophy can be induced by suppressing a factor that inhibits muscle regeneration (Patent Literature 1).

However, it takes a long time to regenerate the muscle by the above mechanism. As a result, conventional treatments that do not take particular measures to promote muscle regeneration cause muscle weakness and delay the return of muscle damaged patients to daily life and sports activities.

Further, if it takes time to regenerate the muscle, part of muscle tissue may be replaced with the scar tissue derived from collagen that has remained in the muscle tissue for a long period of time. Since the scar tissue reduces the strength of plastic muscles, the risk of recurrence of muscle damage is high (Non Patent Literature 1).

Regarding the relationship between the histamine H₃ receptor and muscle, it has been reported that mRNA expression of the histamine H₃ receptor increases with differentiation and maturation of myofibroblasts, and a histamine H₃ receptor agonist that suppresses intracellular calcium influx in electrically stimulated mature myofibroblasts may be involved in the regulation and maintenance mechanism of muscle contraction and relaxation (Non Patent Literature 7), but there is no finding that the muscle regeneration promotion effect by the histamine H₃ receptor agonist has been clarified.

### CITATION LIST

### Patent Literature

Patent Literature 1: WO 2013/039244 A

### Non Patent Literature

Non Patent Literature 1: J. Appl. Physiol., vol. 95, no. 2, pp. 771-780, 2003.
Non Patent Literature 2: Am. J. Sports Med., vol. 27, no. 1, pp. 2-9, 1999.
Non Patent Literature 3: Am. J. Sports Med., vol. 33, no. 5, pp. 745-64, May 2005.
Non Patent Literature 4: J. Bone Joint Surg. Am., vol. 84-A, no. 5, pp. 822-32, May 2002.
Non Patent Literature 5: Dev. Biol., vol. 194, no. 1, pp. 114-128, 1998.
Non Patent Literature 6: J. Cell. Physiol., vol. 138, no. 2, pp. 311-5, February 1989.
Non Patent Literature 7: Eur. J. Pharmacol., vol. 754, pp. 173-8, May 2015

### SUMMARY OF THE INVENTION

### Technical Problem

The conventional treatments that takes time to regenerate the muscle cause muscle weakness easily, decrease motor function of patients and shorten healthy life expectancy, or cause the long-term suspension of activities of athletes easily. Thus a more effective novel treatment has been desired. Further, although the molecular mechanism for muscle regeneration has been widely studied, a drug effective in the muscle regeneration has not been developed yet. Therefore, the development of a drug that promotes muscle regeneration has been strongly desired.

### Solution to Problem

The present inventors performed intensive studies to solve the problems, and as a result, found that muscle regeneration is promoted when a compound having histamine H₃ receptor agonist activity (except for histamine) or a salt thereof is used, and completed the present invention. That is, the present invention relates to the following [1] to [13].
[1] A muscle regeneration promoter, comprising a compound having histamine H₃ receptor agonist activity (except for histamine), or a pharmaceutically acceptable salt thereof.
[2] The muscle regeneration promoter described in the above [1], wherein the compound having histamine H₃ receptor agonist activity is a compound represented by formula (I):

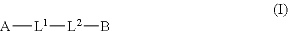

wherein
A is a group represented by
formula (II):

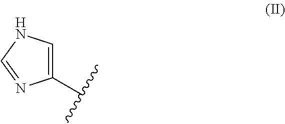 [SMILES: *c1c[nH]cn1]

or
formula (III):

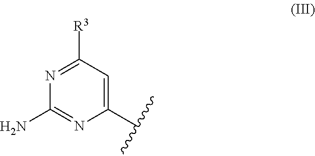

wherein R is a hydrogen atom or a lower alkyl group;
B is a group represented by
formula (IV):

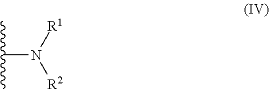

wherein R¹ and R² are each independently a hydrogen atom or a lower alkyl group,
formula (V):

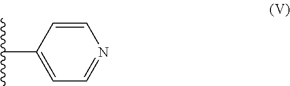 [SMILES: *c1ccncc1]

or
formula (VI):

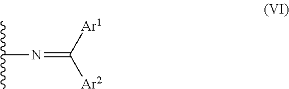

wherein Ar¹ is a phenyl group (the phenyl group may be substituted with one or two substituents selected from the group consisting of a hydroxyl group and a halogen atom); and Ar² is a phenyl group or a heteroaryl group;
L¹ is
a group represented by formula: —C(R^{L1})(R^{L1′})—, or
a group represented by formula: —N(R^{L1})—,
wherein R^{L1} and R^{L1′} are each independently a hydrogen atom or a lower alkyl group;
L² is
a single bond,
a group represented by formula: —C(R^{L2})(R^{L2′})—,
a group represented by formula: —C(R^{L2})(R^{L2′})—C(R^{L3})(R^{L3′})—,
   a group represented by formula: —C(R^{L2})(R^{L2′})—C(R^{L3})(R^{L3′})—C(R^{L4})(R^{L4′})—,
   a group represented by formula: —C(R^{L2})(R^{L2′})—C(R^{L3})(R^{L3′})—C(R^{L4})(R^{L4′})—C(R^{L5})(R^{L5′})—,
   a group represented by formula: —O—C(R^{L2})(R^{L2′})—C(R^{L3})(R^{L3′})—,
   a group represented by formula: —C(R^{L2})(R^{L2′})—C(R^{L3})(R^{L3′})—(C═N(R^{N}))—, or
   a group represented by formula: —C(R^{L2})(R^{L2′})—S—(C═N(R^{N}))—,
   wherein
   R^{L2} and R^{L2′} are each independently a hydrogen atom, a lower alkyl group, or a halo lower alkyl group;
   R^{L3}, R^{L3′}, R^{L4}, R^{L4′}, R^{L5} and R^{L5′} are each independently a hydrogen atom or a lower alkyl group; and
   R^{N} is a hydrogen atom or a lower alkyl group,
   R¹ and R^{L1} may be bonded to each other via a group represented by formula: —(CH₂)ₙ— (wherein n is 1 or 2) to form a 3-membered to 8-membered ring,
   R¹ and R^{L2} may be bonded to each other via a group represented by formula: —(CH₂)ₙ— (wherein n is 1 or 2) to form a 3-membered to 7-membered ring,
   R^{L1} and R^{L2} may be bonded to each other via a group represented by formula: —(CH₂)ₙ— (wherein n is 1 or 2) to form a 3-membered to 5-membered ring,
   R^{L1} and R^{L3} may be bonded to each other via a group represented by formula: —(CH₂)ₙ— (wherein n is 1 or 2) to form a 4-membered to 6-membered ring, and
   R^{L1} and R^{L2′} may be taken together to form a double bond.
[3] The muscle regeneration promoter according to [2], in which
   A is a group represented by formula (II):

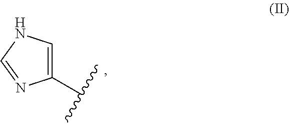 [SMILES: *c1c[nH]cn1]

B is a group represented by formula (IV):

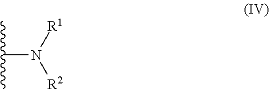

wherein R¹ and R² are each independently a hydrogen atom,
or
a group represented by formula (V):

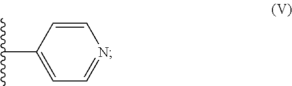 [SMILES: *c1ccncc1]

L¹ is a group represented by formula: —C(R^{L1})(R^{L1′})—, wherein R^{L1} and R^{L1′} are hydrogen atoms; and
L² is
a single bond,
a group represented by formula: —C(R^{L2})(R^{L2′})—, or
a group represented by formula: —C(R^{L2})(R^{L2′})—S—(C═N(R^{N}))—,
wherein R^{L2} and R^{L2′} are each independently a hydrogen atom or a lower alkyl group; and R^{N} is a hydrogen atom.
[4] The muscle regeneration promoter described in the above [1], wherein the compound having histamine H₃ receptor agonist activity is selected from the group consisting of the following compounds <1> to <22>:
<1> (2R)-1-(1H-imidazol-4-yl) propan-2-amine
<2> (2S)-1-chloro-3-(1H-imidazol-4-yl) propan-2-amine
<3> (2R,3R)-3-(1H-imidazol-4-yl) butan-2-amine
<4>2-(1H-imidazol-4-yl)-N-methylethan-1-amine
<5>4-[(1H-imidazol-4-yl)methyl]pyridine
<6>5-(1H-imidazol-4-yl) pentan-1-amine
<7>5-(1H-imidazol-4-yl)-N, N-dimethylpentan-1-amine
<8> 2-(1H-imidazol-4-yl)ethyl carbamimidothioate
<9> 2-(1H-imidazol-4-yl)ethyl N′-methylcarbamimidothioate
<10> 4-(1H-imidazol-4-yl) butanimidamide
<11> 4-[(2R,3S)—2-methylpyrrolidin-3-yl]-1H-imidazole
<12> 4-[(3R,4R)—4-methylpyrrolidin-3-yl]-1H-imidazole
<13> 4-[(1H-imidazol-4-yl)methyl]piperidine
<14> 4-[(pyrrolidin-3-yl)methyl]-1H-imidazole
<15> 4-[(1H-imidazol-4-yl)methyl]-1-methylpiperidine
<16> 1-[(2R,5R)—5-(1H-imidazol-4-yl)oxolan-2-yl]methanamine
<17> (1S,2S)—2-(1H-imidazol-4-yl) cyclopropan-1-amine
<18> 4-[(1H-imidazol-4-yl)methylidene]piperidine
<19> N⁴-(2-aminoethyl)pyrimidine-2,4-diamine
<20> 4-[3-(propylamino) azetidin-1-yl]pyrimidin-2-amine
<21> 2-[(E)-{[(2R)-1-(1H-imidazol-4-yl) propan-2-yl]imino}(phenyl)methyl]phenol
<22> 4-fluoro-2-[(E)-{[(2R)-1-(1H-imidazol-4-yl) propan-2-yl]imino}(1H-pyrrolo-2-yl)methyl]phenol.
[5] The muscle regeneration promoter described in any one of the above [1] to [3], wherein the compound having histamine H₃ receptor agonist activity is a selective histamine H₃ receptor agonist.
[6] The muscle regeneration promoter described in the above [4], wherein the compound having histamine H₃ receptor agonist activity is selected from the group consisting of the following compounds:
<1> (2R)-1-(1H-imidazol-4-yl) propan-2-amine
<5> 4-[(1H-imidazol-4-yl)methyl]pyridine
<8> 2-(1H-imidazol-4-yl)ethyl carbamimidothioate
<13> 4-[(1H-imidazol-4-yl)methyl]piperidine
<15> 4-[(1H-imidazol-4-yl)methyl]-1-methylpiperidine
<20> 4-[3-(propylamino) azetidin-1-yl]pyrimidin-2-amine.
[7] The muscle regeneration promoter described in the above [4], wherein the compound having histamine H₃ receptor agonist activity is selected from the group consisting of the following compounds:
<1> (2R)-1-(1H-imidazol-4-yl) propan-2-amine
<5> 4-[(1H-imidazol-4-yl)methyl]pyridine
<8> 2-(1H-imidazol-4-yl)ethyl carbamimidothioate.
[8] The muscle regeneration promoter described in any one of the above [1] to [7], wherein the muscle regeneration after muscle damage or in myogenic disease is promoted.
[9] The muscle regeneration promoter described in the above [8], wherein the muscle regeneration after muscle damage is promoted.
[10] The muscle regeneration promoter described in the above [9], wherein the muscle damage is muscle strain.
[11] A therapeutic agent for muscle damage, comprising a compound having histamine H₃ receptor agonist activity (except for histamine) or a pharmaceutically acceptable salt thereof.
[12] The therapeutic agent for muscle damage described in the above [11], wherein the compound having histamine H receptor agonist activity is a compound represented by formula (I):

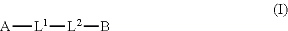

wherein
A is a group represented by
formula (II):

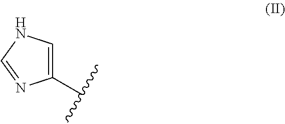 [SMILES: *c1c[nH]cn1]

or
formula (III):

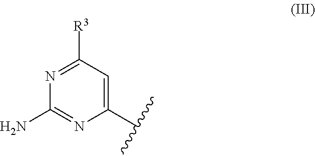

wherein R is a hydrogen atom or a lower allyl group;
B is a group represented by
formula (IV):

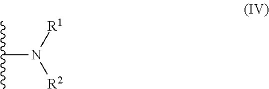

wherein R¹ and R² are each independently a hydrogen atom or a lower alkyl group,
formula (V):

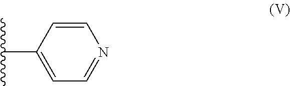 [SMILES: *c1ccncc1]

or
formula (VI):

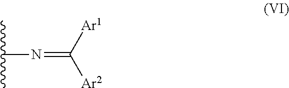

wherein Ar¹ is a phenyl group (the phenyl group may be substituted with one or two substituents selected from the group consisting of a hydroxyl group and a halogen atom); and Ar² is a phenyl group or a heteroaryl group;
L¹ is
a group represented by formula: —C(R^{L1})(R^{L1′})—, or
a group represented by formula: —N(R^{L1})—,
wherein R^{L1} and R^{L1′} are each independently a hydrogen atom or a lower alkyl group;
L² is
a single bond,
a group represented by formula: —C(R^{L2})(R^{L2′})—,
a group represented by formula: —C(R^{L2})(R^{L2′})—C(R^{L3})(R^{L3′})—,
a group represented by formula: —C(R^{L2})(R^{L2′})—C(R^{L3})(R^{L3′})—C(R^{L4})(R^{L4′})—,
a group represented by formula: —C(R^{L2})(R^{L2′})—C(R^{L3})(R^{L3′})—C(R^{L4})(R^{L4′})—C(R^{L5})(R^{L5′})—,
a group represented by formula: —O—C(R^{L2})(R^{L2′})—C(R^{L3})(R^{L3′})—,
a group represented by formula: —C(R^{L2})(R^{L2′})—C(R^{L3})(R^{L3′})—(C═N(R^{N}))—, or
a group represented by formula: —C(R^{L2})(R^{L2′})—S—(C═N(R^{N}))—,
wherein
R^{L2} and R^{L2′} are each independently a hydrogen atom, a lower alkyl group, or a halo lower alkyl group;
R^{L3}, R^{L3′}, R^{L4}, R^{L4′}, R^{L5} and R^{L5′} are each independently a hydrogen atom or a lower alkyl group; and
R^{N} is a hydrogen atom or a lower alkyl group,
R¹ and R^{L1} may be bonded to each other via a group represented by formula: —(CH₂)ₙ— (wherein n is 1 or 2) to form a 3-membered to 8-membered ring,
R¹ and R^{L2} may be bonded to each other via a group represented by formula: —(CH₂)ₙ— (wherein n is 1 or 2) to form a 3-membered to 7-membered ring,
R^{L1} and R^{L2} may be bonded to each other via a group represented by formula: —(CH₂)ₙ— (wherein n is 1 or 2) to form a 3-membered to 5-membered ring,
R^{L1} and R^{L3} may be bonded to each other via a group represented by formula: —(CH₂)ₙ— (wherein n is 1 or 2) to form a 4-membered to 6-membered ring, and
R^{L1} and R^{L2′} may be taken together to form a double bond.
[13] The therapeutic agent for muscle damage described in the above or [12], wherein the muscle damage is a muscle strain.

### Effects of Invention

As shown in Examples to be described later, the muscle regeneration promoter according to the present invention can promote muscle regeneration.

### BRIEF DESCRIPTION OF DRAWINGS

FIG. 1 is a histogram of the regenerated single-muscle fiber area after administration of α-Met His dihydrochloride.

FIG. 2 shows the mean muscle fiber area of regenerated muscle after administration of α-Met His dihydrochloride.

FIG. 3 is a histogram of the regenerated single-muscle fiber area after administration of Imetit dihydrobromide.

FIG. 4 shows the mean muscle fiber area of regenerated muscle after administration of Imetit dihydrobromide.

FIG. 5 is a histogram of the regenerated single-muscle fiber area after administration of Immethridine dihydrobromide.

FIG. 6 shows the mean muscle fiber area of regenerated muscle after administration of Immethridine dihydrobromide.

FIG. 7 is a histogram of the regenerated single-muscle fiber area after administration of N^{α}-methylhistamine dihydrochloride.

FIG. 8 shows the mean muscle fiber area of regenerated muscle after administration of N^{α}-methylhistamine dihydrochloride.

### DESCRIPTION OF EMBODIMENTS

### [Active Ingredient]

The muscle regeneration promoter according to the present invention contains a compound having histamine H₃ receptor agonist activity (except for histamine) or a pharmaceutically acceptable salt thereof as an active ingredient.

The term “histamine H₃ receptor” used herein is one of the receptor subtypes for histamine (also known as 2-(1H-imidazol-4-yl) ethan-1-amine). The histamine H₃ receptor is mainly expressed in nerves, but is also expressed in muscles.

The term “compound having histamine H₃ receptor agonist activity” used herein means a compound that binds to a histamine H₃ receptor and activates the receptor.

However, histamine is excluded from the active ingredient of the muscle regeneration promoter of the present invention.

The term “compound having histamine H₃ receptor agonist activity” used herein is also referred to as a “histamine H₃ receptor agonist”, an “H₃ receptor agonist”, a “histamine H₃ agonist”, or an “H₃ agonist”.

The histamine H₃ receptor agonist may have agonist activity on histamine receptors other than the histamine H₃ receptor (hereinafter, also referred to as “other histamine receptors”). Examples of other histamine receptors include a histamine H₁ receptor, a histamine Hz receptor, and a histamine H₄ receptor.

The histamine H₃ receptor agonist is preferably a “selective histamine H₃ receptor agonist” having an agonist activity selective for the histamine H₃ receptor. This selective agonist is also referred to as “selective H₃ receptor agonist”, “selective histamine H₃ agonist”, or “selective H₃ agonist”).

The “agonist activity selective for the histamine H₃ receptor” refers that the agonist activity for the histamine H₃ receptor is higher (preferably 3 times or more, more preferably 10 times or more, and particularly preferably 30 times or more) than the agonist activity for other histamine receptors.

Methods for measuring agonist activity for the histamine H₃ receptor and other histamine receptors are known, and for example, the agonist activity can be measured and determined in accordance with the test method described in the literature (J. Med. Chem. 2003, 46, 5812-5824; Br. J. Phrmacol. 1994, 112, 847-854; and J. Med. Chem. 2003, 46, 5445-5457).

The histamine H₃ receptor agonist is preferably a compound represented by formula (I):

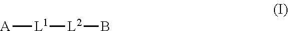

wherein
A is a group represented by
formula (II):

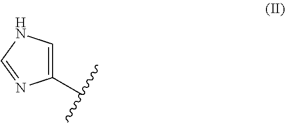 [SMILES: *c1c[nH]cn1]

formula (III):

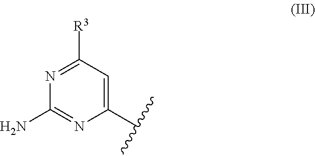

wherein R is a hydrogen atom or a lower alkyl group;
B is a group represented by
formula (IV):

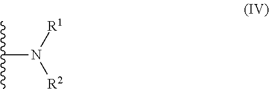

wherein R¹ and R² are each independently a hydrogen atom or a lower alkyl group,
formula (V):

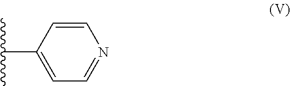 [SMILES: *c1ccncc1]

or
formula (VI):

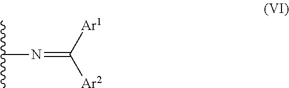

wherein Ar¹ is a phenyl group (the phenyl group may be substituted with one or two substituents selected from the group consisting of a hydroxyl group and a halogen atom); and Ar² is a phenyl group or a heteroaryl group;
L¹ is
a group represented by formula: —C(R^{L1})(R^{L1′})—, or
a group represented by formula: —N(R^{L1})—,
wherein R^{L1} and R^{L1′} are each independently a hydrogen atom or a lower alkyl group;
L² is
a single bond,
a group represented by formula: —C(R^{L2})(R^{L2′})—,
a group represented by formula: —C(R^{L2})(R^{L2′})—C(R^{L3})(R^{L3′})—,
a group represented by formula: —C(R^{L2})(R^{L2′})—C(R^{L3})(R^{L3′})—C(R^{L4})(R^{L4′})—,
a group represented by formula: —C(R^{L2})(R^{L2′})—C(R^{L3})(R^{L3′})—C(R^{L4})(R^{L4′})—C(R^{L5})(R^{L5′})—,
a group represented by formula:—O—C(R^{L2})(R^{L2′})—C(R^{L3})(R^{L3′})—,
a group represented by formula: —C(R^{L2})(R^{L2′})—C(R^{L3})(R^{L3′})—(C═N(R^{N}))—, or
a group represented by formula: —C(R^{L2})(R^{L2′})—S—(C═N(R^{N}))—,
wherein
R^{L2} and R^{L2′} are each independently a hydrogen atom, a lower alkyl group, or a halo lower alkyl group;
R^{L3}, R^{L3′}, R^{L4}, R^{L4′}, R^{L5} and R^{L5′} are each independently a hydrogen atom or a lower alkyl group; and
R^{N} is a hydrogen atom or a lower alkyl group,
R¹ and R^{L1} may be bonded to each other via a group represented by formula: —(CH₂)ₙ— (wherein n is 1 or 2) to form a 3-membered to 8-membered ring,
R¹ and R^{L2} may be bonded to each other via a group represented by formula: —(CH₂)ₙ— (wherein n is 1 or 2) to form a 3-membered to 7-membered ring,
R^{L1} and R^{L2} may be bonded to each other via a group represented by formula: —(CH₂)ₙ— (wherein n is 1 or 2) to form a 3-membered to 5-membered ring,
R^{L1} and R^{L3} may be bonded to each other via a group represented by formula: —(CH₂)ₙ— (wherein n is 1 or 2) to form a 4-membered to 6-membered ring, and
R^{L1} and R^{L2′} may be taken together to form a double bond.

First, terms used in formula (I) will be described.

The “lower alkyl group” means a linear or branched alkyl group having 1 to 6 carbon atoms, and examples thereof include a methyl group, an ethyl group, a propyl group, an isopropyl group, a butyl group, an isobutyl group, a sec-butyl group, a tert-butyl group, a pentyl group, an isopentyl group, an isoamyl group, a neopentyl group, a 1,1-dimethylpropyl group, a 1-methylbutyl group, a 2-methylbutyl group, a 1,2-dimethylpropyl group, a hexyl group, an isohexyl group, a 1-methylpentyl group, a 2-methylpentyl group, a 3-methylpentyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,2,2-trimethylpropyl group, a 1-ethyl-3-methylpropyl group, and the like.

Examples of the “halogen atom” include a fluorine atom, a chlorine atom, a bromine atom, an iodine atom, and the like.

The “halo lower alkyl group” means the “lower alkyl group” in which one or two or more, preferably one to five identical or different halogen atoms are substituted at any substitutable position, and examples thereof include a fluoromethyl group, a difluoromethyl group, a trifluoromethyl group, a 2-fluoroethyl group, a 1,2-difluoroethyl group, a 2,2,2-trifluoroethyl group, a pentafluoroethyl group, a chloromethyl group, a 2-chloroethyl group, a 1,2-dichloroethyl group, a 2,2,2-trichloroethyl group, a bromomethyl group, an iodomethyl group, and the like.

The “any substitutable position” means a site of a substitutable hydrogen atom on a carbon atom, the substitution of which hydrogen atom is chemically accepted, and consequently a stable compound is obtained.

The “heteroaryl group” means a 5-membered or 6-membered monocycle containing, in addition to a carbon atom, one or two or more, preferably one to four heteroatoms that are identically or differently from the group consisting of an oxygen atom, a nitrogen atom and a sulfur atom, or means a bicyclic ring obtained by condensation of the monocycle and a benzene ring or a pyridine ring, and examples thereof include a pyrrolyl group, a furyl group, a thienyl group, an imidazolyl group, a pyrazolyl group, a thiazolyl group, an isothiazolyl group, an oxazolyl group, an isoxazolyl group, a triazolyl group, a tetrazolyl group, a 1,2,3-oxadiazolyl group, a 1,2,4-oxadiazolyl group, a 1,3,4-oxadiazolyl group, a 1,2,5-oxadiazolyl group, a 1,2,3-thiadiazolyl group, a 1,2,4-thiadiazolyl group, a 1,3,4-thiadiazolyl group, a 1,2,5-thiadiazolyl group, a pyridyl group, a pyrazinyl group, a pyrimidinyl group, a pyridazinyl group, a 1,2,4-triazinyl group, a 1,3,5-triazinyl group, an indolyl group, an isoindolyl group, a benzofuranyl group, a benzothienyl group, a benzimidazolyl group, a benzoxazolyl group, a benzisoxazolyl group, a benzothiazolyl group, a benzisothiazolyl group, an indazolyl group, an imidazopyridyl group, a purinyl group, a quinolyl group, a quinolizinyl group, an isoquinolyl group, a phthalazinyl group, a naphthyridinyl group, a quinoxalinyl group, a quinazolinyl group, a cinnolinyl group, a pteridinyl group, a pyrido[3,2-b]pyridyl group, and the like.

Next, various symbols specifying formula (I) will be described in detail with suitable specific examples.

A in formula (I) is a group represented by formula (II):

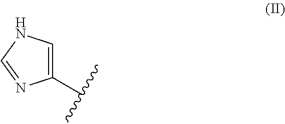 [SMILES: *c1c[nH]cn1]

or
formula (III):

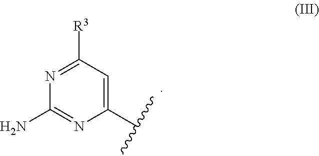

R³ in formula (III) is a hydrogen atom or a lower alkyl group.

Examples of the lower alkyl group of R³ include a methyl group, an ethyl group, a propyl group, an isopropyl group, a butyl group, an isobutyl group, a sec-butyl group, a tert-butyl group, and the like, and a methyl group, an ethyl group, and an isopropyl group are preferable.

Preferable examples of R³ include a hydrogen atom, a methyl group, an ethyl group, and an isopropyl group.

B in formula (I) is a group represented by formula (IV):

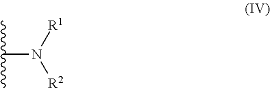

formula (V):

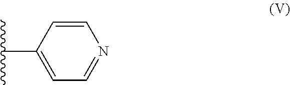 [SMILES: *c1ccncc1]

or
formula (VI):

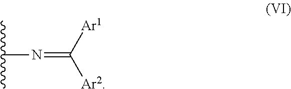

R¹ and R² in formula (IV) are each independently a hydrogen atom or a lower alkyl group.

Examples of the lower alkyl group of R¹ and R² include a methyl group, an ethyl group, a propyl group, an isopropyl group, a butyl group, an isobutyl group, a sec-butyl group, a tert-butyl group, and the like, and a methyl group is preferable.

Preferable examples of R¹ and R² include a hydrogen atom and a methyl group.

Ar¹ in formula (VI) is a phenyl group. This phenyl group may be substituted with one or two substituents selected from the group consisting of a hydroxyl group and a halogen atom.

The “phenyl group that may be substituted with one or two substituents selected from the group consisting of a hydroxyl group and a halogen atom” means an unsubstituted phenyl group, a phenyl group substituted with one or two hydroxyl groups, a phenyl group substituted with one or two halogen atoms, or a phenyl group substituted with one hydroxyl group and one halogen atom, examples thereof include a phenyl group, a 2-hydroxyphenyl group, a 3-hydroxyphenyl group, a 4-hydroxyphenyl group, a 2-fluorophenyl group, a 3-fluorophenyl group, a 4-fluorophenyl group, a 2-chlorophenyl group, a 3-chlorophenyl group, a 4-chlorophenyl group, a 2-fluoro-3-hydroxyphenyl group, a 2-fluoro-4-hydroxyphenyl group, a 2-fluoro-5-hydroxyphenyl group, a 2-fluoro-6-hydroxyphenyl group, a 3-fluoro-2-hydroxyphenyl group, a 3-fluoro-4-hydroxyphenyl group, a 3-fluoro-5-hydroxyphenyl group, a 5-fluoro-2-hydroxyphenyl group, a 4-fluoro-2-hydroxyphenyl group, a 4-fluoro-3-hydroxyphenyl group, and the like, and a 2-hydroxyphenyl group and a 5-fluoro-2-hydroxyphenyl group are preferable.

Preferable examples of Ar¹ include a phenyl group, a 2-hydroxyphenyl group, and a 5-fluoro-2-hydroxyphenyl group.

Ar² in formula (VI) is a phenyl group or a heteroaryl group.

Examples of the heteroaryl group include a pyrrolyl group, a furyl group, a thienyl group, a pyridyl group, a pyrimidinyl group, and the like, and a pyrrolyl group is preferable.

Preferable examples of Ar² include a phenyl group and a pyrrolyl group.

L¹ in formula (I) is a group represented by formula: —C(R^{L1})(R^{L1′})— or a group represented by formula: —N(R^{L1})—.

R^{L1} and R^{L1′} in each formula are each independently a hydrogen atom or a lower alkyl group.

Examples of the lower alkyl group include a methyl group, an ethyl group, a propyl group, an isopropyl group, a butyl group, an isobutyl group, a sec-butyl group, a tert-butyl group, and the like, and a methyl group is preferable.

Preferable examples of R^{L1} and R^{L1′} include a hydrogen atom and a methyl group.

L² in formula (I) is a group selected from the following (i) to (viii):
(i) a single bond,
(ii) a group represented by formula: —C(R^{L2})(R^{L2′})—,
(iii) a group represented by formula: —C(R^{L2})(R^{L2′})—C(R^{L3})(R^{L3′})—,
(iv) a group represented by formula: —C(R^{L2})(R^{L2′})—C(R^{L3})(R^{L3′})—C(R^{L4})(R^{L4′})—,
(v) a group represented by formula: —C(R^{L2})(R^{L2′})—C(R^{L3})(R^{L3′})—C(R^{L4})(R^{L4′})—C(R^{L5})(R^{L5′})—,
(vi) a group represented by formula:—O—C(R^{L2})(R^{L2′})—C(R^{L3})(R^{L3′})—,
(vii) a group represented by formula: —C(R^{L2})(R^{L2′})—C(R^{L3})(R^{L3′})—(C═N(R^{N}))—,
(viii) a group represented by formula: —C(R^{L2})(R^{L2′})—S—(C═N(R^{N}))—

R^{L2} and R^{L2′} in each formula are each independently a hydrogen atom, a lower alkyl group, or a halo lower alkyl group.

Examples of the lower alkyl group include a methyl group, an ethyl group, a propyl group, an isopropyl group, a butyl group, an isobutyl group, a sec-butyl group, a tert-butyl group, and the like, and a methyl group is preferable.

Examples of the halo lower alkyl group include a fluoromethyl group, a difluoromethyl group, a trifluoromethyl group, a chloromethyl group, and the like, and a chloromethyl group is preferable.

Preferable examples of R^{L2} and R^{L2′} include a hydrogen atom, a methyl group, and a chloromethyl group.

R^{L3}, R^{L3′}, R^{L4}, R^{L4′}, R^{L5} and R^{L5′} in each formula are each independently a hydrogen atom or a lower alkyl group.

Examples of the lower alkyl group include a methyl group, an ethyl group, a propyl group, an isopropyl group, a butyl group, an isobutyl group, a sec-butyl group, a tert-butyl group, and the like, and a methyl group is preferable.

Preferable examples of R^{L3}, R^{L3′}, R^{L4}, R^{L4′}, R^{L5} and R^{L5′} include a hydrogen atom and a methyl group, and a hydrogen atom is more preferable.

R^{N} in each formula is a hydrogen atom or a lower alkyl group.

Examples of the lower alkyl group include a methyl group, an ethyl group, a propyl group, an isopropyl group, a butyl group, an isobutyl group, a sec-butyl group, a tert-butyl group, and the like, and a methyl group is preferable.

Preferable examples of R^{N} include a hydrogen atom and a methyl group.

R¹ and R^{L1} may be bonded to each other via a group represented by formula: —(CH₂)ₙ— (wherein n is 1 or 2) to form a 3-membered to 8-membered ring. Examples of the compound of formula (I) in this case include compounds represented by formula (I-10) or formula (I-11).

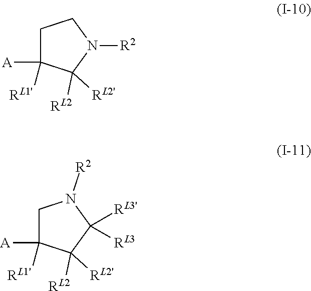

R¹ and R^{L2} may be bonded to each other via a group represented by formula: —(CH)— (wherein n is 1 or 2) to form a 3-membered to 7-membered ring. Examples of the compound of formula (I) in this case include compounds represented by formula (I-12) or formula (I-13).

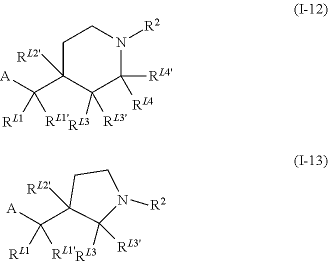

R^{L1} and R^{L2} may be bonded to each other via a group represented by formula: —(CH₂)ₙ— (wherein n is 1 or 2) to form a 3-membered to 5-membered ring. Examples of the compound of formula (I) in this case include compounds represented by formula (I-14) or formula (I-15).

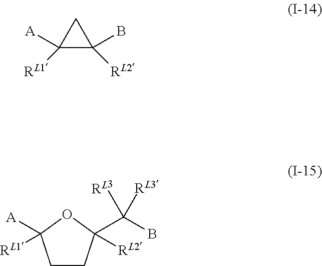

R^{L1} and R^{L3} may be bonded to each other via a group represented by formula: —(CH₂)ₙ— (wherein n is 1 or 2) to form a 4-membered to 6-membered ring. Examples of the compound of formula (I) in this case include compounds represented by formula (I-16).

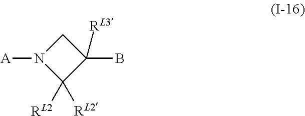

R^{L1} and R^{L2′} may be taken together to form a double bond. This indicates that two adjacent carbon atoms in which R^{L1} and R^{L2′} are substituted are bonded to each other by a double bond, and is represented by, for example, the following formula (I-17).

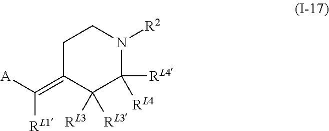

In formula (I-17), R¹ and R^{L2} are bonded via a group represented by formula: —(CH₂)ₙ— (wherein n is 2) to form a 6-membered ring.

Next, a combination of -L¹-L²- of formula (I) will be exemplified.
(1) When L¹ is a group represented by formula: —C(R^{L1})(R^{L1′})—, and L² is a single bond, formula (I) is represented as formula (I-1).

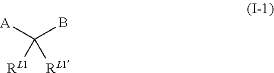

(2) When L¹ is a group represented by formula: —C(R^{L1})(R^{L1′})—, and L² is a group represented by formula: —C(R^{L2})(R^{L2′})—, formula (I) is represented as formula (I-2).

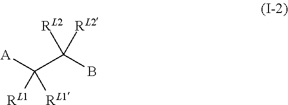

(3) When L¹ is a group represented by formula: —C(R^{L1})(R^{L1′})—, and L² is a group represented by formula: —C(R^{L2})(R^{L2′})—C(R^{L3})(R^{L3′})—, formula (I) is represented as formula (I-3).

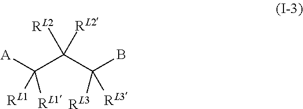

(4) When L¹ is a group represented by formula: —C(RM) (R^{L1′})—, and L² is a group represented by formula: —C(R^{L2})(R^{L2′})—C(R^{L3})(R^{L3′})—C(R^{L4})(R^{L4′})—, formula (I) is represented as formula (I-4).

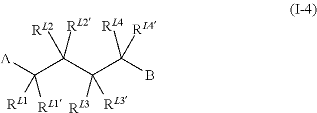

(5) When L¹ is a group represented by formula: —C(R^{L1})(R^{L1′})—, and L² is a group represented by formula: —C(R^{L2})(R^{L2′})—C(R^{L3}) (R^{L3′})—C(R^{L4})(R^{L4′})—C(R^{L5})(R^{L5′})—, formula (I) is represented as formula (I-5).

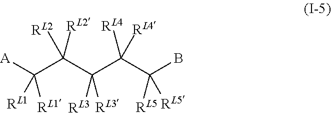

(6) When L¹ is a group represented by formula: —C(R^{L1})(R^{L1′})—, and L² is a group represented by formula: — O—C(R^{L2})(R^{L2′})—C(R^{L3})(R^{L5′})—, formula (I) is represented as formula (I-6).

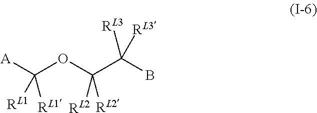

(7) When L¹ is a group represented by formula: —C(R^{L1})(R^{L1′})—, and L² is a group represented by formula: —C(R^{L2})(R^{L2′})—C(R^{L3})(R^{L5′})—(C═N(R^{N}))—, formula (I) is represented as formula (I-7).

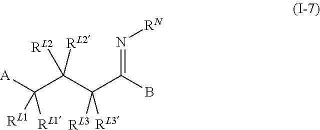

(8) When L¹ is a group represented by formula: —C(R^{L1})(R^{L1′})—, and L² is a group represented by formula: —C(R^{L2})(R^{L2′})—S—(C═N(R^{N}))—, formula (I) is represented as formula (I-8).

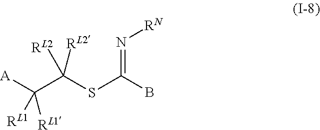

(9) When L¹ is a group represented by formula: —N(R^{L1})—, and L² is a group represented by formula: —C(R^{L2})(R^{L2′})—C(R^{L3})(R^{L5′})—, formula (I) is represented as formula (I-9).

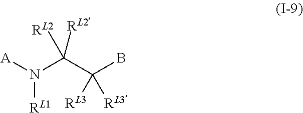

Preferred examples of the compound represented by formula (I) include compounds <I> to <02> as shown in the following Table 1.

TABLE 1

| | Structure | Compound Name | Other Name |
|---|---|---|---|
| 1 | 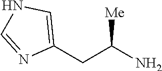 | (2R)-1-(1H- imidazol-4- yl)propan-2-amine | R-α- methylhisatmine |
| 2 | 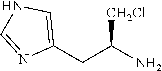 | (2S)-1-chloro-3- (1H-imidazol-4- yl)propan-2-amine | (S)-α- chloromethylhistamine |
| 3 | 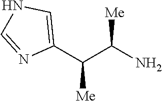 | (2R,3R)-3-(1H- imidazol-4- yl)butan-2-amine | R-α-S-β- dimethylhistamine |
| 4 | 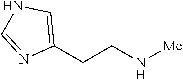 | 2-(1H-imidazol-4- yl)-N-methylethan- 1-amine | N^{α}- methylhistamine |
| 5 | 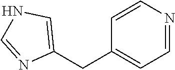 [SMILES: c1cc(Cc2c[nH]cn2)ccn1] | 4-[(1H-imidazol-4- yl)methyl]pyridine | immethridine |
| 6 | 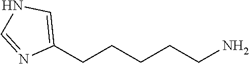 [SMILES: NCCCCCc1c[nH]cn1] | 5-(1H-imidazol-4- yl)pentan-1-amine | impentamine |
| 7 | 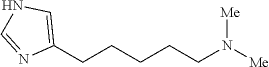 [SMILES: CN(C)CCCCCc1c[nH]cn1] | 5-(1H-imidazol-4- yl)-N,N- dimethylpentan-1- amine | VUF5207 |
| 8 | 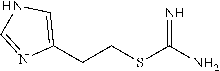 [SMILES: N=C(N)SCCc1c[nH]cn1] | 2-(1H-imidazol-4- yl)ethyl carbamimidothioate | imetit |
| 9 | 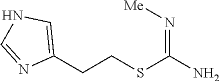 [SMILES: C/N=C(/N)SCCc1c[nH]cn1] | 2-(1H-imidazol-4- yl)ethyl N′- methylcarbamimidothioate | |
| 10 | 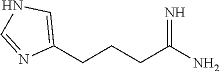 [SMILES: N=C(N)CCCc1c[nH]cn1] | 4-(1H-imidazol-4- yl)butanimidamide | SKF91606 |
| 11 | 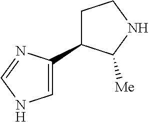 [SMILES: C[C@H]1NCC[C@@H]1c1c[nH]cn1] | 4-[(2R,3S)-2- methylpyrrolidin-3- yl]-1H-imidazole | Immepyr |
| 12 | 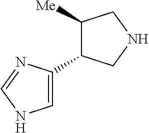 [SMILES: C[C@H]1CNC[C@@H]1c1c[nH]cn1] | 4-[(3R,4R)-4- methylpyrrolidin-3- yl]-1H-imidazole | Sch-50971 |
| 13 | 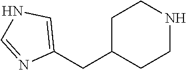 [SMILES: c1nc(CC2CCNCC2)c[nH]1] | 4-[(1H-imidazol-4- yl)methyl]piperidine | immepip |
| 14 | 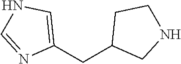 [SMILES: c1nc(CC2CCNC2)c[nH]1] | 4-[(pyrrolidin-3- yl)methyl]-1H- imidazole | VUF4848 |
| 15 | 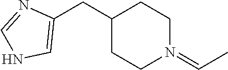 | 4-[(1H-imidazol-4- yl)methyl]-1- methylpiperidine | Methimepip |
| 16 | 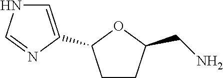 [SMILES: NC[C@H]1CC[C@H](c2c[nH]cn2)O1] | 1-[(2R,5R)-5-(1H- imidazol-4- yl)oxolan-2- yl]methanamine | imifuramine |
| 17 | 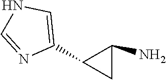 | (1S,2S)-2-(1H- imidazol-4- yl)cyclopropan-1- amine | VUF5297 |
| 18 | 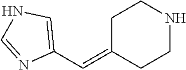 [SMILES: C(=C1CCNCC1)c1c[nH]cn1] | 4-[(1H-imidazol-4- yl)methylidene] piperidine | VUF5510 |
| 19 | 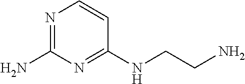 [SMILES: NCCNc1ccnc(N)n1] | N⁴-(2- aminoethyl)pyrimidine- 2,4-diamine | |
| 20 | 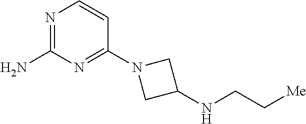 [SMILES: CCCNC1CN(c2ccnc(N)n2)C1] | 4-[3- (propylamino)azetidin- 1-yl]pyrimidin- 2-amine | VUF16839 |
| 21 | 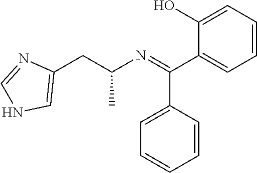 [SMILES: C[C@H](Cc1c[nH]cn1)/N=C(\c1ccccc1)c1ccccc1O] | 2-[(E)-{[(2R)-1- (1H-imidazol-4- yl)propan-2- yl]imino}(phenyl) methyl]phenol | BP 2-94 |
| 22 | 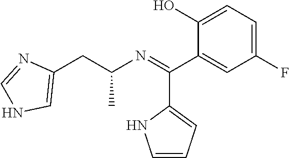 [SMILES: C[C@H](Cc1c[nH]cn1)/N=C(\c1ccc[nH]1)c1cc(F)ccc1O] | 4-fluoro-2-[(E)- {[(2R)-1-(1H- imidazol-4- yl)propan-2- yl]imino) (1H- pyrrolo-2- yl)methyl]phenol | |

The relationship between compounds <1> to <22> listed in the Table 1 and formula (I) is shown in the following Table 2.

TABLE 2

| | A | L¹ | R^{L1} | R^{L1′} | L² | R^{L2} | R^{L2} | B | R¹ | R² |
|---|---|---|---|---|---|---|---|---|---|---|
| <1> | 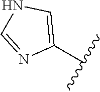 [SMILES: *c1c[nH]cn1] | —C(R^{L1})(R^{L1′})— | H | H | —C(R^{L2})(R^{L2′})— | CH₃ | H | 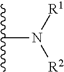 | H | H |
| <2> | 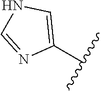 [SMILES: *c1c[nH]cn1] | —C(R^{L1})(R^{L1′})— | H | H | —C(R^{L2})(R^{L2′})— | CH₂Cl | H | 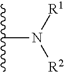 | H | H |
| <3> | 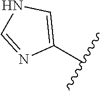 [SMILES: *c1c[nH]cn1] | —C(R^{L1})(R^{L1′})— | CH₃ | H | —C(R^{L2})(R^{L2′})— | CH₃ | H | 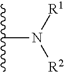 | H | H |
| <4> | 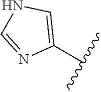 [SMILES: *c1c[nH]cn1] | —C(R^{L1})(R^{L1′})— | H | H | —C(R^{L2})(R^{L2′})— | H | H | 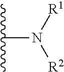 | CH₃ | H |
| <5> | 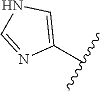 [SMILES: *c1c[nH]cn1] | —C(R^{L1})(R^{L1′})— | H | H | Single bond | — | — | 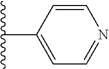 [SMILES: *c1ccncc1] | — | — |

| | | | | | R^{L2}, R^{L2′}, | | | |
|---|---|---|---|---|---|---|---|---|
| | | | | | R^{L3}, R^{L3′}, | | | |
| | | | R^{L1} | | R^{L4}, R^{4′}, | | | |
| | A | L¹ | R^{L1′} | L² | R^{L5}, R^{L5′} | R^{N} | B | R¹, R² |
| <6> | 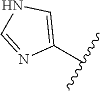 [SMILES: *c1c[nH]cn1] | —C(R^{L1})(R^{L1′})— | H | —C(R^{L2})(R^{L2′})—C(R^{L3})(R^{L3′})— C(R^{L4})(R^{L4′})—C(R^{L5})(R^{L5′})— | H | — | 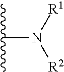 | H |
| <7> | 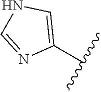 [SMILES: *c1c[nH]cn1] | —C(R^{L1})(R^{L1′})— | H | —C(R^{L2})(R^{L2′})—C(R^{L3})(R^{L3′})— C(R^{L4})(R^{L4′})—C(R^{L5})(R^{L5′})— | H | — | 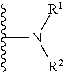 | CH₃ |
| <8> | 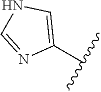 [SMILES: *c1c[nH]cn1] | —C(R^{L1})(R^{L1′})— | H | —C(R^{L2})(R^{L2′})—S—(C═N(R^{N}))— | H | H | 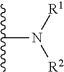 | H |
| <9> | 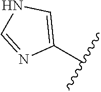 [SMILES: *c1c[nH]cn1] | —C(R^{L1})(R^{L1′})— | H | —C(R^{L2})(R^{L2′})—S—(C═N(R^{N}))— | H | CH₃ | 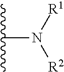 | H |
| <10> | 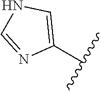 [SMILES: *c1c[nH]cn1] | —C(R^{L1})(R^{L1′})— | H | —C(R^{L2})(R^{L2′})—C(R^{L3})(R^{L3′})— (C═N(R^{N}))— | H | H | 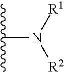 | H |

| | | | | | | R^{L1′}, R^{L2′}, | | | |
|---|---|---|---|---|---|---|---|---|---|
| | | | | | | R^{L3}, R^{L3′}, | | | |
| | A | L¹ | R^{L1} | L² | R^{L2} | R^{L4}, R^{L4′} | B | R¹ | R² |
| <11> | 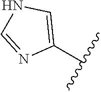 [SMILES: *c1c[nH]cn1] | —C(R^{L1})(R^{L1′})— | — | —C(R^{L2})(R^{L2′})— | CH₃ | H | 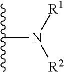 | R¹ and R^{L1}: —CH₂—CH₂— | H |
| <12> | 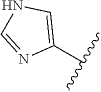 [SMILES: *c1c[nH]cn1] | —C(R^{L1})(R^{L1′})— | — | —C(R^{L2})(R^{L2′})—C(R^{L3})(R^{L3′})— | CH₃ | H | 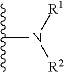 | R¹ and R^{L1}: —CH₂— | H |
| <13> | 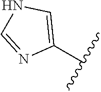 [SMILES: *c1c[nH]cn1] | —C(R^{L1})(R^{L1′})— | H | —C(R^{L2})(R^{L2′})—C(R^{L3})(R^{L3′})— C(R^{L4})(R^{L4′})— | — | H | 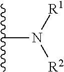 | R¹ and R^{L2}: —CH₂—CH₂— | H |
| <14> | 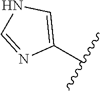 [SMILES: *c1c[nH]cn1] | —C(R^{L1})(R^{L1′})— | H | —C(R^{L2})(R^{L2′})—C(R^{L3})(R^{L3′})— | — | H | 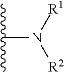 | R¹ and R^{L2}: —CH₂—CH₂— | H |
| <15> | 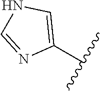 [SMILES: *c1c[nH]cn1] | —C(R^{L1})(R^{L1′})— | H | —C(R^{L2})(R^{L2′})—C(R^{L3})(R^{L3′})— C(R^{L4})(R^{L4′})— | — | H | 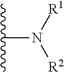 | R¹ and R^{L2}: —CH₂—CH₂— | CH₃ |

| | | | | | R^{L1′}, | | | | |
|---|---|---|---|---|---|---|---|---|---|
| | | | | | R^{L2}, R^{L2′}, | | | | |
| | | | | | R^{L3}, R^{L3′}, | | | | |
| | A | L¹ | R^{L1} | L² | R^{L4}, R^{L4′} | B | R¹ | R² | R³ |
| <16> | 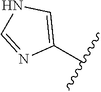 [SMILES: *c1c[nH]cn1] | —C(R^{L1})(R^{L1′})— | R^{L1} and R^{L2}: —CH₂—CH₂— | —O—C(R^{L2})(R^{L2′})— C(R^{L3})(R^{L3′})— | H | 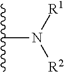 | H | H | — |
| <17> | 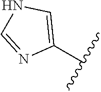 [SMILES: *c1c[nH]cn1] | —C(R^{L1})(R^{L1′})— | R^{L1} and R^{L2}: —CH₂— | —C(R^{L2})(R^{L2′})— | H | 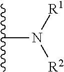 | H | H | — |
| <18> | 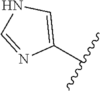 [SMILES: *c1c[nH]cn1] | —C(R^{L1})(R^{L1′})— | R^{L1} and R^{L2′}: Double bond | —C(R^{L2})(R^{L2′})—C(R^{L3})(R^{L3′})— C(R^{L4})(R^{L4′})— | H | 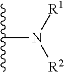 | R¹ and R^{L2}: —CH₂—CH₂— | H | — |
| <19> | 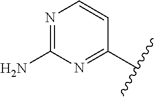 [SMILES: *c1ccnc(N)n1] | —N(R^{L1})— | H | —C(R^{L2})(R^{L2′})—C(R^{L3})(R^{L3′})— | H | 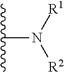 | H | H | H |
| <20> | 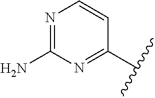 [SMILES: *c1ccnc(N)n1] | —N(R^{L1})— | R^{L1} and R^{L3}: —CH₂— | —C(R^{L2})(R^{L2′})—C(R^{L3})(R^{L3′})— | H | 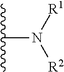 | CH₂CH₂CH₃ | H | H |

| | A | L¹ | R^{L1} | R^{L1′} | L² | R^{L2} | R^{L2′} | B | Ar¹ | Ar² |
|---|---|---|---|---|---|---|---|---|---|---|
| <21> | 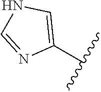 [SMILES: *c1c[nH]cn1] | —C(R^{L1})(R^{L1′})— | H | H | —C(R^{L2})(R^{L2′})— | CH₃ | H | 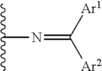 | 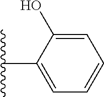 [SMILES: *c1ccccc1O] | 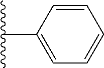 [SMILES: *c1ccccc1] |
| <22> | 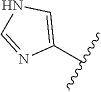 [SMILES: *c1c[nH]cn1] | —C(R^{L1})(R^{L1′})— | H | H | —C(R^{L2})(R^{L2′})— | CH₃ | H | 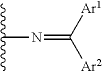 | 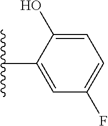 [SMILES: *c1cc(F)ccc1O] | 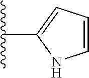 [SMILES: *c1ccc[nH]1] |

Among the compounds listed in Table 1, the following compounds are selective histamine H₃ receptor agonists.
<1> (2R)-1-(1H-imidazol-4-yl) propan-2-amine
<5> 4-[(1H-imidazol-4-yl)methyl]pyridine
<8> 2-(1H-imidazol-4-yl)ethyl carbamimidothioate
<13> 4-[(1H-imidazol-4-yl)methyl]piperidine
<15> 4-[(1H-imidazol-4-yl)methyl]-1-methylpiperidine
<20> 4-[3-(propylamino) azetidin-1-yl]pyrimidin-2-amine

The literature (The Journal of Pharmacology and Experimental Therapeutics, 1992, 263, 304-310) discloses that (2R)-1-(1H-imidazol-4-yl) propan-2-amine (R-α-methylhistamine) is a selective histamine H₃ receptor agonist.

The literature (J. Med. Chem. 2004, 47, 2414-2417) discloses that 4-[(1H-imidazol-4-yl)methyl]pyridine (immethridine) is a selective histamine H₃ receptor agonist.

The literature (The Journal of Pharmacology and Experimental Therapeutics, 1992, 263, 304-310) discloses that 2-(1H-imidazol-4-yl)ethyl carbamimidothioate (imetit) is a selective histamine H₃ receptor agonist.

The literature (J. Med. Chem. 1994, 37, 332-333) discloses that 4-[(1H-imidazol-4-yl)methyl]piperidine (immepip) is a selective histamine H₃ receptor agonist.

The literature (J. Med. Chem. 2005, 48, 2100-2107) discloses that 4-[(1H-imidazol-4-yl)methyl]-1-methylpiperidine (Methimepip) is a selective histamine H₃ receptor agonist.

The literature (J. Med. Chem. 2019, 62, 10848-10866) discloses that 4-[3-(propylamino) azetidin-1-yl]pyrimidin-2-amine (VUF16839) is a selective histamine H₃ receptor agonist.

The compound represented by formula (I) may have an asymmetric center, a chiral axis, or a chiral plane.

Some chemical structures in Table 1 are depicted using bold lines or dashed lines to represent chemical bonds. These bold lines and dashed lines depict absolute stereochemistry. A bold line indicates that a substituent is above the plane of the carbon atom to which it is attached, and a dashed line indicates that a substituent is below the plane of the carbon atom to which it is attached.

The compound represented by formula (I) may be generated as a racemates, as a racemic mixture, or as an individual diastereomer.

Both optical isomers of the compound represented by formula (I) and mixtures thereof are included in the histamine H₃ receptor agonists used in the present invention.

The compound represented by formula (I) may exist as a tautomer. Even if only one tautomeric structure is described herein, both tautomeric forms, including the other tautomeric structure, are included in the histamine H₃ receptor agonists used in the present invention.

For example, imidazole, which is a partial structure of the histamine H₃ receptor agonist used in the present invention, exists as a tautomer represented by the following formula. Both of these tautomers are included in the histamine H₃ receptor agonists used in the present invention.

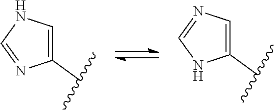 [SMILES: *c1c[nH]cn1 ; *c1cnc[nH]1]

As the pharmaceutically acceptable salt of a histamine H₃ receptor agonist (hereinafter, also referred to as the “salt thereof”), for example, a hydrochloride, a hydrobromide, a maleate, a fumarate, an oxalate, a tartrate, etc. are mentioned.

The pharmaceutically acceptable salt of the histamine H₃ receptor agonist include solvate with a pharmaceutically acceptable solvent such as water or ethanol.

The histamine H₃ receptor agonist and a salt thereof are known substances, and are easily available on the market, or easily synthesized by a combination of known synthesis reactions.

### [Muscle Regeneration Promoter]

The term “muscle regeneration promotion” used herein means that the regeneration of the damaged muscle tissue caused by muscle damage, myogenic disease, or the like is promoted.

As the muscle damage, for example, muscle strain (caused by external force (for example, a bruise or the like)), pulled muscle (caused by internal force such as sudden contraction of muscle), and cervical sprain (so-called whiplash injury), are mentioned.

As the myogenic disease, for example, muscular dystrophy, and distal myopathy, etc. are mentioned. In this regard, muscle damage and myogenic disease are common to each other in that muscle regeneration compensating for necrosis of muscle fibers (muscle damage) occurs.

The concentration of the histamine H₃ receptor agonist or a salt thereof in a muscle regeneration promoter can be appropriately set depending on the degree of muscle damage and the like.

The muscle regeneration promoter can be applied to an animal having muscles without any limitation. The application target is preferably a mammal (a human, or a non-human mammal (for example, a horse or a cow)), and more preferably a human. Further, there are no restrictions on the sex and age of the application target.

### [Pharmaceutical Formulation]

The muscle regeneration promoter can be provided as a pharmaceutical formulation. The pharmaceutical formulation includes an oral formulation and a parenteral formulation. As the oral formulation, for example, a tablet, a capsule, a powder, or a granule can be mentioned. As the parenteral formulation, for example, a sterilized pharmaceutical formulation in a liquid state such as solution or suspension, specifically, an injection or an infusion can be mentioned. The pharmaceutical formulation is preferably an oral formulation, but in a case of the parenteral formulation, an intramuscular injection is preferred.

The pharmaceutical formulation may contain a pharmaceutically acceptable carrier or diluent together with an active ingredient. The formulation can be conducted by using a common formulation technique.

As the “pharmaceutically acceptable carrier or diluent”, for example, an excipient (for example, fat, beeswax, polyol of semi-solid or liquid, or natural or hardened oil); water (for example, distilled water, particularly, distilled water for injection); physiological saline; alcohol (for example, ethanol); glycerol; a polyol; an aqueous solution of glucose; mannitol; plant oil; and an additive agent (for example, a bulking agent, a disintegrant, a binding agent, a lubricant, a wetting agent, a stabilizer, an emulsifier, a dispersant, a preservative, a sweetener, a coloring agent, a seasoning or an aromatic substance, a thickener, a diluent, a buffer substance, a solvent, a solubilizer, a drug for achieving a storage effect, a salt for changing an osmotic pressure, a coating agent, or an antioxidant), etc. are mentioned.

The muscle regeneration promoter can be applied to various forms of pharmaceutical formulations. As the various forms, for example, an oral formulation (a tablet, a capsule, a powder, a granule, or a solution), a parenteral formulation (a sterilized solution or a suspension), a suppository, an ointment, etc. are mentioned.

The pharmaceutical formulation may be a solid formulation, or may also be a liquid formation.

The solid formulation can be produced as it is in the form of a tablet, a capsule, a granule, or a powder, but can also be produced by using an appropriate carrier (additive). As the carrier (additive), for example, a saccharide (for example, lactose, or glucose); a starch (for example, maize, wheat, or rice); a fatty acid (for example, stearic acid); an inorganic salt (for example, magnesium aluminometasilicate, or anhydrous calcium phosphate); a synthetic polymer (for example, polyvinyl pyrrolidone, or polyalkylene glycol); a fatty acid salt (for example, calcium stearate, or magnesium stearate); an alcohol (for example, stearyl alcohol, or benzyl alcohol); a synthetic cellulose derivative (for example, methyl cellulose, carboxymethyl cellulose, ethyl cellulose, or hydroxypropyl methyl cellulose); and other usually-used additives (gelatin, talc, plant oil, and gum arabic), etc. are mentioned.

The solid preparation can contain, for example, 0.1 to 100% by mass, preferably 5 to 98% by massof an active ingredient based on the total pharmaceutical formulation.

The liquid formulation can be produced in the form of a suspension, a syrup, an injection, an infusion (intravenous infusion), or the like by using an appropriate additive usually used in a liquid formulation (for example, water, an alcohol, or plant-derived oil such as soybean oil, peanut oil, sesame oil).)

As the appropriate solvent or diluent in a case of parenteral administration in the form of intramuscular injection, intravenous injection, or subcutaneous injection, for example, distilled water for injection, a lidocaine hydrochloride aqueous solution (for intramuscular injection), a saline solution, an aqueous solution of glucose, ethanol, polyethylene glycol, propylene glycol, a liquid for intravenous injection (for example, an aqueous solution of citric acid, sodium citrate, or the like), an electrolyte solution (for intravenous drip infusion or intravenous injection), and a mixed solution thereof, etc. are mentioned

These injections may be prepared in the form of pre-dissolved active ingredient, and further may be prepared in the form that is dissolved at the time of use as a powder of the active ingredient as it is or a power of the active ingredient added with an appropriate carrier (additive). The injection can contain, for example, 0.005 to 25% by mass of an active ingredient based on the total pharmaceutical formulation.

### [Therapeutic Agent for Muscle Damage]

The histamine H₃ receptor agonist and a salt thereof can treat muscle damage by promoting the regeneration of the damaged muscle tissue. Accordingly, the muscle regeneration promoter according to the present invention can be grasped also as a therapeutic agent for muscle damage.

The description about the active ingredient and formulation of the muscle regeneration promoter is applied to the therapeutic agent for muscle damage.

### EXAMPLES

Next, the effects of the present invention will be specifically described by way of Examples, however, the present invention is not limited to these Examples.

### Experimental Method

### 1. Evaluation Compounds

The following four kinds of compounds were evaluated.

| Example | Compound name | Note |
|---|---|---|
| 1 | R-α-Methylhistamine | Compound 1 in Table 1 |
| | dihydrochloride | |
| 2 | Imetit dihydrobromide | Compound 8 in Table 1 |
| 3 | Immethridine | Compound 5 in Table 1 |
| | dihydrobromide | |
| 4 | N^{α}-Methylhistamine | Compound 4 in Table 1 |
| | dihydrochloride | |

### 2. Test Animals

Seven-week old C57BL/6 Male mice (CLEA Japan, Inc.) were purchased, and used for experiment at the age of 8 weeks.

### 3. Muscle-Damage Model Animal

A model animal to which muscle damage had been caused by administration of snake venom cardiotoxin (CTX) was used. The muscle-damaged model animal has been widely used in studies on the regeneration from muscle damage.

Under the anesthesia with isoflurane, 50 μL of 10 μM CTX was administered to the tibialis anterior muscle of the right hindlimb of the mouse. After 7 days of the administration of CTX, the tibialis anterior muscle was collected by dissection, and supplied to the preparation of a muscle tissue section.

### 4. Preparation of Muscle Tissue Section

Immediately after the collection of the tibialis anterior muscle, the tibialis anterior muscle was immersed in isopentane cooled with liquid nitrogen and was rapidly frozen. The frozen muscle tissue was cut into slices each having a thickness of 10 μm by using a cryostat (Leica Biosystems), and the slice was attached onto an antistripping coated slide glass (Matsunami Glass Ind., Ltd.).

### 5. Immunofluorescence Staining of Muscle Tissue Section

A muscle tissue section was sufficiently air dried for 30 minutes under room temperature. After that, the muscle tissue section was fixed by immersing it in acetone cooled to −30° C. and treating at −30° ° C. for 20 minutes. The fixed section was air dried once and washed with PBS. Then the section was blocked by dropwisely adding a blocking reagent (Blocking One, NACALAI TESQUE, INC.) to the section and being subjected to the blocking treatment for 1 hour. Next, a primary antibody (Anti-laminin-2 (α-2 Chain) antibody, Rat monoclonal (Sigma-Aldrich)) obtained by being diluted 500 times with the blocking reagent was added dropwise, and the reaction was conducted at overnight at 4° C. Since laminin to which a primary antibody binds is a protein expressed in all muscle cells, the primary antibody was used in this experiment in order to measure the area of individual muscle cells in a section. The muscle tissue section after the reaction with the primary antibody was washed with PBS, and then was reacted for 1 hour with a secondary antibody (CF 488A Goat Anti-Rat IgG (H+L) (Biotium)) obtained by being diluted 500 times with the blocking reagent. The secondary antibody that is an anti-rat antibody conjugated with a fluorescent dye binds to the primary antibody, and stains the laminin. The muscle tissue section after the reaction with the secondary antibody was washed with PBS, and sealed by using “VECTASHIELD Hard. Set with DAPI” (Vector), and then the fluorescence observation was performed with an inverted microscope FSX100 (Olympus). The “VECTASHIELD Hard Set with DAPI” was used for staining the central nucleus of muscle cells.

### 6. Evaluation of Muscle Regeneration

The muscle regeneration was evaluated on the basis of the image data taken from the fluorescence observation. In this experiment, muscle cells each having a central nucleus (single muscle fiber having a central nuclei) were used as an indicator for regenerated muscle. After the image data was taken into image analysis software ImageJ (NIH), a muscle cell having a central nucleus was extracted. The cross-sectional area of the extracted individual cells was measured on the basis of the cell membrane stained with laminin. For the area measurement, “Analyze Particles” that is an add-in analysis program on ImageJ was used. The measurement results were shown as a distribution chart (histogram) of the areas and number of regenerated single-muscle fibers, and as an average value of the cross-sectional areas of all the regenerated single muscle fibers (mean muscle fiber area).

### Example 1: Muscle Regeneration Promotion Effect of R-α-Methylhistamine Dihydrochloride

R-α-Methylhistamine ((2R)-1-(1H-imidazol-4-yl) propan-2-amine) is a selective histamine H₃ receptor agonist.

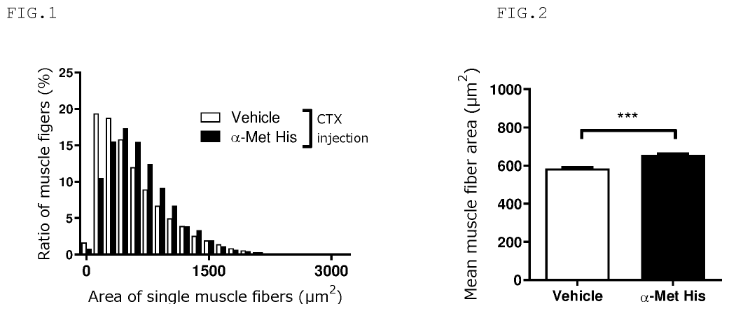

R-α-Methylhistamine dihydrochloride (Sigma-Aldrich) dissolved in a PBS with 5% tween 20 at a concentration of 6.3 mM was injected intramuscularly in a volume of 10 μL into the tibialis anterior muscle of both legs of the mouse once a day from the day before CTX administration to the day before dissection (6 days after CTX administration). In a control group (Vehicle), a PBS with 5% tween 20 solution was injected intramuscularly into the tibialis anterior muscle of both legs of a mouse. The number and area of regenerated single muscle fibers in the collected tibialis anterior muscle were measured by the above method. The results are shown in FIGS. 1 and 2.

When muscle regeneration was evaluated on the basis of the histogram of the single-muscle fiber area, the histogram of the R-α-methylhistamine dihydrochloride (α-Met His) administration group was shifted to the right side (in a direction in which the area becomes larger) as compared with the histogram of the Vehicle administration group (FIG. 1).

When muscle regeneration was evaluated on the basis of the mean muscle fiber area, a significant increase in the mean muscle fiber area (12.3%) was observed in the R-α-methylhistamine dihydrochloride administration group as compared with the Vehicle administration group (FIG. 2. ***P<0.0001).

These results indicate that R-α-methylhistamine dihydrochloride promoted an increase of the area of muscle regenerated from the damage due to CTX administration, that is, muscle regeneration.

### Example 2: Muscle Regeneration Promotion Effect of Imetit Dihydrobromide

Imetit (2-(1H-imidazol-4-yl)ethyl carbamimidothioate) is a selective histamine H₃ receptor agonist.

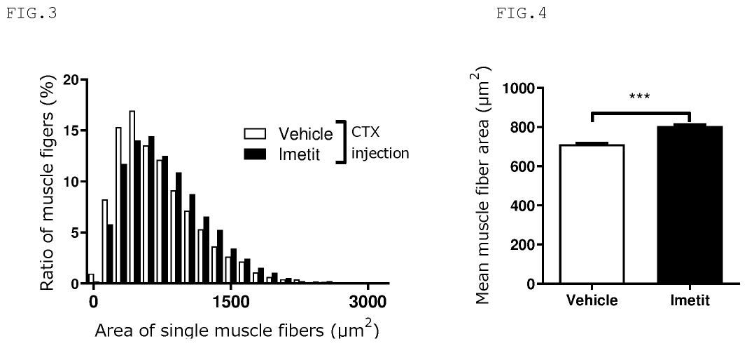

Imetit dihydrobromide (Tocris) dissolved in PBS at a concentration of 1 μM was injected intramuscularly in a volume of 10 μL into the tibialis anterior muscle of both legs of a mouse once a day from the day before CTX administration to the day before dissection (6 days after CTX administration). In a control group (Vehicle), a PBS was injected intramuscularly into the tibialis anterior muscle of both legs of the mouse. The number and area of regenerated single muscle fibers in the collected tibialis anterior muscle were measured by the above method. The results are shown in FIGS. 3 and 4.

When the muscle regeneration was evaluated on the basis of the histogram of the single-muscle fiber area, the histogram of the Imetit dihydrobromide (Imetit) administration group was shifted to the right side (in a direction in which the area becomes larger) as compared with the histogram of the Vehicle administration group (FIG. 3).

When the muscle regeneration was evaluated on the basis of the mean muscle fiber area, a significant increase in the mean muscle fiber area (13.18) was observed in the Imetit dihydrobromide administration group as compared with the Vehicle administration group (FIG. 4. ***P<0.0001).

These results indicate that imetit dihydrobromide promoted the increase of the area of muscle regenerated from the damage due to CTX administration, that is, muscle regeneration.

### Example 3: Muscle Regeneration Promotion Effect of Immethridine Dihydrobromide

Immethridine (4-[(1H-imidazol-4-yl)methyl]pyridine) is a selective histamine Ha receptor agonist.

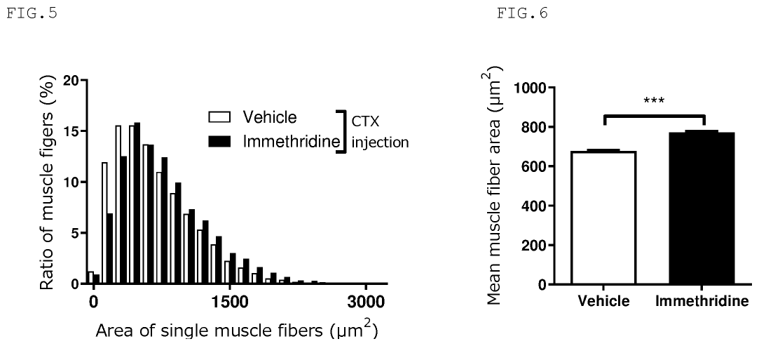

Immethridine dihydrobromide (Santa Cruz Biotechnology) dissolved in a PBS at a concentration of 1 μM was injected intramuscularly in a volume of 10 μL into the tibialis anterior muscle of both legs of a mouse once a day from the day before CTX administration to the day before dissection (6 days after CTX administration). In a control group (Vehicle), a PBS was injected intramuscularly into the tibialis anterior muscle of both legs of a mouse. The number and area of regenerated single muscle fibers in the collected tibialis anterior muscle were measured by the above method. The results are shown in FIGS. 5 and 6.

When the muscle regeneration was evaluated on the basis of the histogram of the single-muscle fiber area, the histogram of the Immethridine dihydrobromide (Immethridine) administration group was shifted to the right side (in a direction in which the area becomes larger) as compared with the histogram of the Vehicle administration group (FIG. 5).

When the muscle regeneration was evaluated on the basis of the mean muscle fiber area, a significant increase in the mean muscle fiber area (14.0%) was observed in the Immethridine dihydrobromide administration group as compared with the Vehicle administration group (FIG. 6. ***P<0.0001).

These results indicate that Immethridine dihydrobromide promoted the increase of the area of muscle regenerated from the damage due to CTX administration, that is, muscle regeneration.

### Example 4: Muscle Regeneration Promotion Effect of N^{α}-Methylhistamine Dihydrochloride

N^{α}-Methylhistamine (2-(1H-imidazol-4-yl)-N-methylethan-1-amine) is a non-selective histamine H₃ receptor agonist that has high selectivity for the histamine H₃ receptor but also has agonist activity for the histamine H₁ and Hz receptors (Pharmacol. Rev., vol. 42, no. 1, pp. 45-83, 1990).

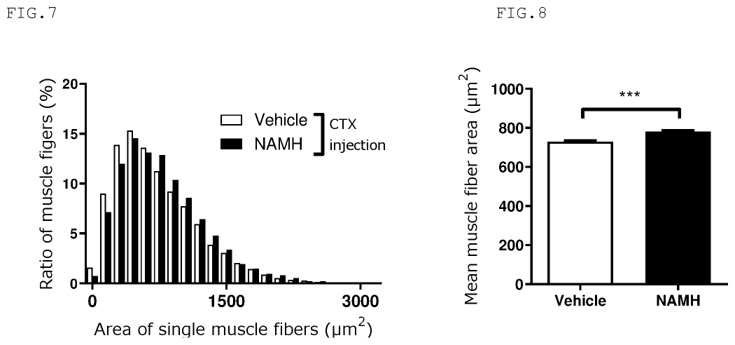

N^{α}-Methylhistamine dihydrochloride (Sigma-Aldrich) dissolved in PBS at a concentration of 1 μM was injected intramuscularly in a volume of 10 μL into the tibialis anterior muscle of both legs of a mouse once a day from the day before CTX administration to the day before dissection (6 days after CTX administration). In a control group (Vehicle), a PBS was injected intramuscularly into the tibialis anterior muscle of both legs of a mouse. The number and area of regenerated single muscle fibers in the collected tibialis anterior muscle were measured by the above method. The results are shown in FIGS. 7 and 8.

When the muscle regeneration was evaluated on the basis of the histogram of the single-muscle fiber area, the histogram of the No-methylhistamine dihydrochloride (NAMH) administration group was shifted to the right side (in a direction in which the area becomes larger) as compared with the histogram of the Vehicle administration group (FIG. 7).

When the muscle regeneration was evaluated on the basis of the mean muscle fiber area, a significant increase in the mean muscle fiber area (7.1%) was observed in the N^{α}-methylhistamine dihydrochloride administration group as compared with the Vehicle administration group (FIG. 8. ***P<0.0001).

These results indicate that N^{α}-methylhistamine dihydrochloride promoted the increase of the area of muscle regenerated from the damage due to CTX administration, that is, muscle regeneration.

### INDUSTRIAL APPLICABILITY

By using the muscle regeneration promoter according to the present invention, the return to daily life and sports activities of a patients with muscle damage can be accelerated. Therefore, the present invention can be used in the treatment of muscle damage.

1. A method for promoting muscle regeneration in an animal, comprising a step of administering a compound having histamine H₃ receptor agonist activity (except for histamine) or a pharmaceutically acceptable salt thereof to the animal.

2. The method according to claim 1, wherein the compound having histamine H₃ receptor agonist activity is a compound represented by formula (I): 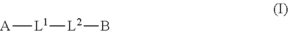
wherein
A is a group represented by
formula (II): 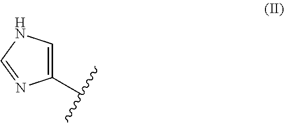 [SMILES: *c1c[nH]cn1]
or
formula (III): 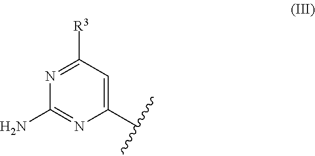
wherein R³ is a hydrogen atom or a lower alkyl group;
B is a group represented by
formula (IV): 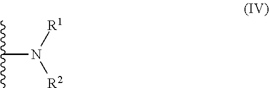
wherein R¹ and R² are each independently a hydrogen atom or a lower alkyl group,
formula (V): 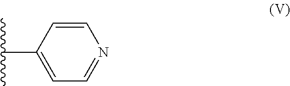 [SMILES: *c1ccncc1]
or
formula (VI): 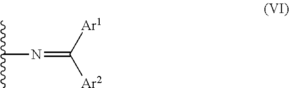
wherein Ar¹ is a phenyl group (the phenyl group may be substituted with one or two substituents selected from the group consisting of a hydroxyl group and a halogen atom); and Ar² is a phenyl group or a heteroaryl group;
L¹ is
a group represented by formula: —C(R^{L1})(R^{L1′})—, or
a group represented by formula: —N(R^{L1})—,
wherein R^{L1} and R^{L1′} are each independently a hydrogen atom or a lower alkyl group;
L² is
a single bond,
a group represented by formula: —C(R^{L2})(R^{L2′})—,
a group represented by formula: —C(R^{L2})(R^{L2′})—C(R^{L3})(R^{L3′})—,
a group represented by formula: —C(R^{L2})(R^{L2′})—C(R^{L3})(R^{L3′})—C(R^{L4})(R^{L4′})—,
a group represented by formula: —C(R^{L2})(R^{L2′})—C(R^{L3})(R^{L3′})—C(R^{L4})(R^{L4′}), C(R^{L5})(R^{L5′})—,
a group represented by formula: —O—C(R^{L2})(R^{L2′})—C(R^{L3})(R^{L3′})—,
a group represented by formula: —C(R^{L2})(R^{L2′})—C(R^{L3})(R^{L3′})—(C═N(R^{N}))—, or
a group represented by formula: —C(R^{L2})(R^{L2′})—S—(C═N(R^{N}))—,
wherein
R^{L2} and R^{L2′} are each independently a hydrogen atom, a lower alkyl group, or a halo lower alkyl group;
R^{L3}, R^{L3′}, R^{L4}, R^{L4′}, R^{L5} and R^{L5′} are each independently a hydrogen atom or a lower alkyl group; and
R^{N} is a hydrogen atom or a lower alkyl group,
R¹ and R^{L1} may be bonded to each other via a group represented by formula: —(CH₂)ₙ— (wherein n is 1 or 2) to form a 3-membered to 8-membered ring,
R¹ and R^{L2} may be bonded to each other via a group represented by formula: —(CH₂)ₙ— (wherein n is 1 or 2) to form a 3-membered to 7-membered ring,
R^{L1} and R^{L2} may be bonded to each other via a group represented by formula: —(CH₂)ₙ— (wherein n is 1 or 2) to form a 3-membered to 5-membered ring,
R^{L1} and R^{L3} may be bonded to each other via a group represented by formula: —(CH₂)ₙ— (wherein n is 1 or 2) to form a 4-membered to 6-membered ring, and
R^{L1} and R^{L2′} may be taken together to form a double bond.

3. The method according to claim 2, wherein
A is a group represented by formula (II): 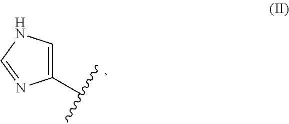 [SMILES: *c1c[nH]cn1]
B is a group represented by formula (IV): 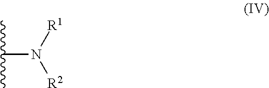
wherein R¹ and R² are each independently a hydrogen atom,
or
a group represented by formula (V): 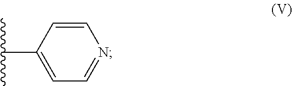 [SMILES: *c1ccncc1]
L¹ is a group represented by formula: —C(R^{L1})(R^{L1′})—, wherein R^{L1} and R^{L1′} are hydrogen atoms; and
L² is
a single bond,
a group represented by formula: —C(R^{L2})(R^{L2′})—, or
a group represented by formula: —C(R^{L2})(R^{L2′})—S—(C═N(R^{N}))—,
wherein R^{L2} and R^{L2′} are each independently a hydrogen atom or a lower alkyl group; and R^{N} is a hydrogen atom.

4. The method according to claim 1, wherein the compound having histamine H₃ receptor agonist activity is selected from the group consisting of the following compounds <1> to <22>:
<1> (2R)-1-(1H-imidazol-4-yl)propan-2-amine
<2> (2S)-1-chloro-3-(1H-imidazol-4-yl)propan-2-amine
<3> (2R,3R)-3-(1H-imidazol-4-yl)butan-2-amine
<4> 2-(1H-imidazol-4-yl)-N-methylethan-1-amine
<5> 4-[(1H-imidazol-4-yl)methyl]pyridine
<6> 5-(1H-imidazol-4-yl)pentan-1-amine
<7> 5-(1H-imidazol-4-yl)-N,N-dimethylpentan-1-amine
<8> 2-(1H-imidazol-4-yl)ethyl carbamimidothioate
<9> 2-(1H-imidazol-4-yl)ethyl N′-methylcarbamimidothioate
<10> 4-(1H-imidazol-4-yl)butanimidamide
<11> 4-[(2R,3S)-2-methylpyrrolidin-3-yl]-1H-imidazole
<12> 4-[(3R,4R)-4-methylpyrrolidin-3-yl]-1H-imidazole
<13> 4-[(1H-imidazol-4-yl)methyl]piperidine
<14> 4-[(pyrrolidin-3-yl)methyl]-1H-imidazole
<15> 4-[(1H-imidazol-4-yl)methyl]-1-methylpiperidine
<16> 1-[(2R,5R)-5-(1H-imidazol-4-yl)oxolan-2-yl]methanamine
<17> (1S,2S)-2-(1H-imidazol-4-yl)cyclopropan-1-amine
<18> 4-[(1H-imidazol-4-yl)methylidene]piperidine
<19> N⁴-(2-aminoethyl)pyrimidine-2,4-diamine
<20> 4-[3-(propylamino)azetidin-1-yl]pyrimidin-2-amine
<21> 2-[(E)-{[(2R)-1-(1H-imidazol-4-yl)propan-2-yl]imino}(phenyl)methyl]phenol
<22> 4-fluoro-2-[(Z)-{[(2R)-1-(1H-imidazol-4-yl)propan-2-yl]imino}(1H-pyrrolo-2-yl)methyl]phenol.

5. The method according to claim 1, wherein the compound having histamine H₃ receptor agonist activity is a selective histamine H₃ receptor agonist.

6. The method according to claim 4, wherein the compound having histamine H₃ receptor agonist activity is selected from the group consisting of the following compounds:
<1> (2R)-1-(1H-imidazol-4-yl)propan-2-amine
<5> 4-[(1H-imidazol-4-yl)methyl]pyridine
<8> 2-(1H-imidazol-4-yl)ethyl carbamimidothioate
<13> 4-[(1H-imidazol-4-yl)methyl]piperidine
<15> 4-[(1H-imidazol-4-yl)methyl]-1-methylpiperidine
<20> 4-[3-(propylamino)azetidin-1-yl]pyrimidin-2-amine.

7. The method according to claim 4, wherein the compound having histamine H₃ receptor agonist activity is selected from the group consisting of the following compounds:
<1> (2R)-1-(1H-imidazol-4-yl)propan-2-amine
<5> 4-[(1H-imidazol-4-yl)methyl]pyridine
<8> 2-(1H-imidazol-4-yl)ethyl carbamimidothioate.

8. The method according to claim 1, wherein muscle regeneration after muscle damage or in myogenic disease is promoted.

9. The method according to claim 8, wherein the muscle regeneration after muscle damage is promoted.

10. The method according to claim 9, wherein the muscle damage is muscle strain.

11. A method for treating muscle damage in an animal, comprising a step of administering a compound having histamine H₃ receptor agonist activity (except for histamine), or a pharmaceutically acceptable salt thereof to the animal.

12. The method according to claim 11, wherein the compound having histamine H₃ receptor agonist activity is a compound represented by formula (I): 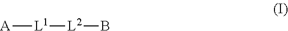
wherein
A is a group represented by
formula (II): 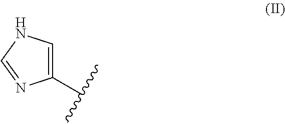 [SMILES: *c1c[nH]cn1]
or
formula (III): 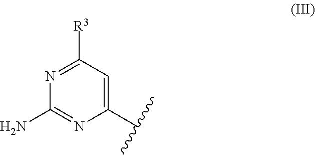
wherein R³ is a hydrogen atom or a lower alkyl group;
B is a group represented by
formula (IV): 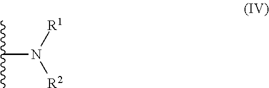
wherein R¹ and R² are each independently a hydrogen atom or a lower alkyl group,
formula (V): 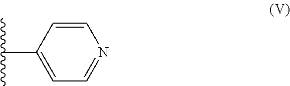 [SMILES: *c1ccncc1]
or
formula (VI): 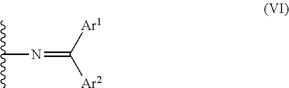
wherein Ar¹ is a phenyl group (the phenyl group may be substituted with one or two substituents selected from the group consisting of a hydroxyl group and a halogen atom); and Ar² is a phenyl group or a heteroaryl group;
L¹ is
a group represented by formula: —C(R^{L1})(R^{L1′})—, or
a group represented by formula: —N(R^{L1})—,
wherein R^{L1} and R^{L1′} are each independently a hydrogen atom or a lower alkyl group;
L² is
a single bond,
a group represented by formula: —C(R^{L2})(R^{L2′})—,
a group represented by formula: —C(R^{L2})(R^{L2′})—C(R^{L3})(R^{L3′})—,
a group represented by formula: —C(R^{L2})(R^{L2′})—C(R^{L3})(R^{L3′})—C(R^{L4})(R^{L4′})—,
a group represented by formula: —C(R^{L2})(R^{L2′})—C(R^{L3})(R^{L3′})—C(R^{L4})(R^{L4′})—C(R^{L5})(R^{L5′})—,
a group represented by formula: —O—C(R^{L2})(R^{L2′})—C(R^{L3})(R^{L3′})—,
a group represented by formula: —C(R^{L2})(R^{L2′})—C(R^{L3})(R^{L3′})—(C═N(R^{N}))—, or
a group represented by formula: —C(R^{L2})(R^{L2′})—S—(C═N(R^{N}))—,
wherein
R^{L2} and R^{L2′} are each independently a hydrogen atom, a lower alkyl group, or a halo lower alkyl group;
R^{L3}, R^{L3′}, R^{L4}, R^{L4′}, R^{L5} and R^{L5′} are each independently a hydrogen atom or a lower alkyl group; and
R^{N} is a hydrogen atom or a lower alkyl group,
R¹ and R^{L1} may be bonded to each other via a group represented by formula: —(CH₂)ₙ— (wherein n is 1 or 2) to form a 3-membered to 8-membered ring,
R¹ and R^{L2} may be bonded to each other via a group represented by formula: —(CH₂)ₙ— (wherein n is 1 or 2) to form a 3-membered to 7-membered ring,
R^{L1} and R^{L2} may be bonded to each other via a group represented by formula: —(CH₂)ₙ— (wherein n is 1 or 2) to form a 3-membered to 5-membered ring,
R^{L1} and R^{L3} may be bonded to each other via a group represented by formula: —(CH₂)ₙ— (wherein n is 1 or 2) to form a 4-membered to 6-membered ring, and
R^{L1} and R^{L2′} may be taken together to form a double bond.

13. The method according to claim 11, wherein the muscle damage is muscle strain.
